(19) United States
(12) Patent Application Publication
(10) Pub. No.: US 2007/0244193 A1
(43) Pub. Date: Oct. 18, 2007

(54) Intramuscular antiviral treatments

(76) Inventors: Yarlagadda S. Babu, Birmingham, AL (US); Pooran Chand, Birmingham, AL (US); Shanta Bantia, Birmingham, AL (US); Shane Arnold, Hoover, AL (US)

Correspondence Address: VIKSNINS HARRIS & PADYS PLLP, P.O. BOX 111098, ST. PAUL, MN, 55111-1098, US

(21) Appl. No.: 11/476,401

(22) Filed: Jun. 28, 2006

Related U.S. Application Data
(63) Continuation of application No. PCT/US06/13535, filed on Apr. 12, 2006.

(51) Int. Cl. A61K 31/195

(52) U.S. Cl. 514/563

(57) ABSTRACT

The invention provides unit dosage forms, kits, and methods useful for treating viral infections.

### RELATED APPLICATIONS

This patent document claims priority to International Application No. PCT/US 2006/013535, filed Apr. 12, 2006, which application is incorporated herein by reference in its entirety.

### BACKGROUND

The influenza virus neuraminidase inhibitor peramivir has marked activity against the influenza virus in vitro and in experimentally infected mice (Govorkova et al., Antimicrobial Agents and Chemotherapy, 45(10), 2723-2732 (2001); and Smee et al., Antimicrobial Agents and Chemotherapy, 45(3), 743-748 (2001)). Unfortunately, clinical trials using peramivir showed an undesirably low inhibitory effect on influenza in humans following oral administration over a period of days. Currently there remains a need for methods and formulations that are useful for treating viral infections such as influenza infections.

### SUMMARY OF CERTAIN EMBODIMENTS OF THE INVENTION

It has unexpectedly been discovered that a single intramuscular administration of peramivir to a mouse is effective to treat influenza. These findings are unexpected not only because of the high effectiveness of a single administration of the compound, but also because of the low dose of the compound that was found to provide effective treatment. The ability to obtain therapeutically useful effects with a single administration is important, inter alia, because it minimizes patient compliance issues that result from the need for multiple administrations. Additionally, the administration of a low dose is important because it minimizes cost and the potential for side-effects.

Accordingly, in one embodiment the invention provides a method for treating a viral infection (e.g., an influenza infection) in a human comprising administering an effective anti-viral amount of a compound of formula I, II, III, or IV:

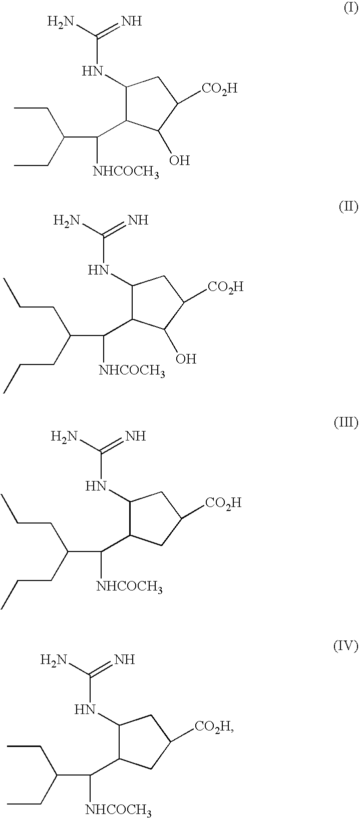

or a pharmaceutically acceptable salt thereof, to the human by an intramuscular route.

The invention also provides a method for inhibiting a neuraminidase in a human comprising administering an effective inhibitory amount of a compound of formula I, II, III, or IV, or a pharmaceutically acceptable salt thereof, to the human by an intramuscular route.

The invention also provides a unit dosage form that is suitable for intramuscular administration to a human comprising up to about 500 mgs (e.g., about 150 mg) of a compound of formula I, II, III, or IV, or a pharmaceutically acceptable salt thereof.

The invention also provides a kit comprising packaging materials, a compound of formula I, II, III, or IV, or a pharmaceutically acceptable salt thereof, and instructions for administering the compound to a human by an intramuscular route.

The invention also provides the use of a compound of formula I, II, III, or IV, or a pharmaceutically acceptable salt thereof, in the manufacture of a medicament for intramuscular injection for increasing life expectancy and/or reducing mortality in a group of mammals, e.g., humans, exposed to a source of an influenza virus, by intramuscular injection of a dose, e.g., an effective antiviral dose, of the medicament into each member of the group presenting clinical symptoms of infection.

The invention also provides the use of a compound of formula I, II, III, or IV, or a pharmaceutically acceptable salt thereof, in the manufacture of a medicament for intramuscular injection for increasing life expectancy or reducing mortality in a group of mammals, e.g., humans, exposed to a source of an influenza virus, by intramuscular injection of a dose of the medicament into each member of the group.

### DETAILED DESCRIPTION

The influenza virus neuraminidase inhibitor peramivir has been previously shown to have marked activity against influenza virus in vitro and in experimentally infected mice (Govorkova et al., (2001); and Smee et al., (2001)). Unfortunately, clinical trials using this drug showed an inadequate inhibitory effect on influenza in humans.

It has been discovered that a single intramuscular injection of peramivir significantly reduces weight loss and mortality in mice infected with influenza A/H1N1. A single intramuscular injection of peramivir can thus be used to treat influenza infections and to provide an alternate option to oseltamivir during an influenza outbreak.

Peramivir was tested as a single intramuscular injection in the mouse influenza model and was found to be active when administered intramuscularly. In three different studies of a prophylaxis model using two different strains (H1N1 and H3N2) of influenza A virus, efficacy of a single intramuscular injection of peramivir was compared to oral treatment (q.d.×5 days) of either oseltamivir or peramivir. Although 5 days (b.i.d.) of oseltamivir is normally used in the clinic for treatment of influenza, once daily dosing for 5 days was also shown to be effective. In all three studies, the efficacy of a single intramuscular injection of peramivir at doses of 10 or 20 mg/kg was comparable to the oral treatment (q.d.×5 days) of oseltamivir or peramivir at the same dose in terms of survival, mean days to death, and weight loss. At doses of 2 mg/kg, as a single intramuscular injection, peramivir demonstrated comparable efficacy in terms of survival. Nevertheless, the maximum weight loss was greater in the single intramuscular peramivir-treated group versus the oral (q.d.×5 days) oseltamivir-treated group. The maximum weight loss for the treatment groups was observed around days 8-10. It should be noted that while the lowest dose of peramivir (1 mg/kg) was not effective in terms of survival, there was a significant increase in the mean days to death. Peramivir was highly effective in mice with a viral challenge that caused 70% lethality when treatment was initiated as late as 48 hours post-infection.

Single intramuscular injections of either peramivir or oseltamivir were also compared in the H1N1 mouse influenza model. The survival data indicated that a single intramuscular injection of peramivir is efficacious and provides complete protection against lethality. Conversely, a single intramuscular injection of oseltamivir provided no significant protection against lethality. The weight loss data is consistent with the survival data and indicates that a single intramuscular injection of peramivir is effective in preventing weight loss in infected mice, unlike the oseltamivir group. These studies indicate that peramivir is efficacious when given as a single intramuscular injection, whereas oseltamivir is not effective by the same route of administration in the mouse influenza model. A single intramuscular injection of oseltamivir carboxylate in mice showed a similar effect as the single intramuscular injection of oseltamivir.

The IC₅₀s of peramivir and oseltamivir carboxylate are subnanomolar against H1N1 at 0.11 and 0.69 nM (Bantia et al., Antimicrob. Agents Chemother. 45, 1162-1167 (2001)) and H3N2 at 0.59 and 0.55 nM, respectively. In spite of similar potency against neuraminidase enzymes, a single intramuscular injection of oseltamivir (carboxylate) is not effective. However, peramivir as a single intramuscular injection is superior to oseltamivir (carboxylate) given as single intramuscular injections. Although the slow off-rate of peramivir was demonstrated with the N9 neuraminidase, one would expect peramivir to bind tightly to both N1 and N2 neuraminidases since the amino acid residues in the active site are highly conserved among different neuraminidase subtypes.

In summary, peramivir is a potent inhibitor of neuraminidase activity. Prophylactic and delayed single intramuscular administrations were effective in preventing lethality and weight loss in the mouse influenza model. In view of the in vivo and in vitro data, peramivir is effective as a single intramuscular injection and can be used in the treatment of human influenza virus infections.

Accordingly, in one embodiment the invention provides a method for treating a viral infection in a human comprising administering an effective amount of a compound of formula I, II, III, or IV, or a pharmaceutically acceptable salt thereof, to the human by intramuscular administration. Typically, the effective amount is administered in a single intramuscular administration. The methods of the invention provide for high patient compliance as they involve a low dose of the effective agent.

In one embodiment of the invention, the effective inhibitory amount of the compound of formula I, II, III, or IV is up to about 500 mg (e.g., from about 10 mg to about 500 mg).

In one embodiment of the invention, the effective inhibitory amount of the compound of formula I, II, III, or IV is up to about 150 mg.

In one embodiment of the invention, the effective inhibitory amount of the compound of formula I, II, III, or IV is about 150 mg.

According to the methods of the invention, a compound of formula I, II, III, or IV is administered to a human intramuscularly. In one embodiment of the invention, the compound of formula I, II, III, or IV is administered once to a human intramuscularly. In another embodiment of the invention, a neuraminidase inhibitor is also administered to the human orally. In one embodiment of the invention, the neuraminidase inhibitor that is administered orally is oseltamivir carboxylate. In one embodiment of the invention, the neuraminidase inhibitor that is administered orally is a compound of formula I, II, III, or IV, or a pharmaceutically acceptable salt thereof. In one embodiment of the invention, the neuraminidase inhibitor that is administered orally is a compound of formula Ia, IIa, IIIa, or IVa:

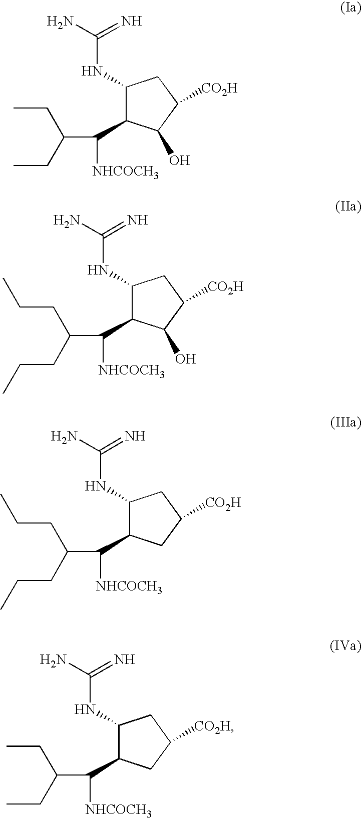

or a pharmaceutically acceptable salt thereof. In one embodiment of the invention, the neuraminidase inhibitor that is administered orally is a compound of formula Ia, or a pharmaceutically acceptable salt thereof.

According to the methods of the invention, the compound of formula I, II, III, or IV, or a pharmaceutically acceptable salt thereof, can also be administered in combination with one or more additional therapeutic agents, such as anti-viral agents (e.g., agents active against influenza) or antibiotics.

The intramuscular formulations of the invention can also comprise one or more additional therapeutic agents, such as anti-viral agents (e.g., agents active against influenza) and antibiotics.

The compounds used in the invention are known in the art and can be synthesized by the art worker using available methods (see, e.g., U.S. Pat. No. 6,562,861).

Specific values listed herein for radicals, substituents, and ranges, are for illustration only; they do not exclude other defined values or other values within defined ranges for the radicals and substituents

A specific compound of formula I, II, III, or IV is a compound of formula Ia, IIa, IIIa, or IVa:

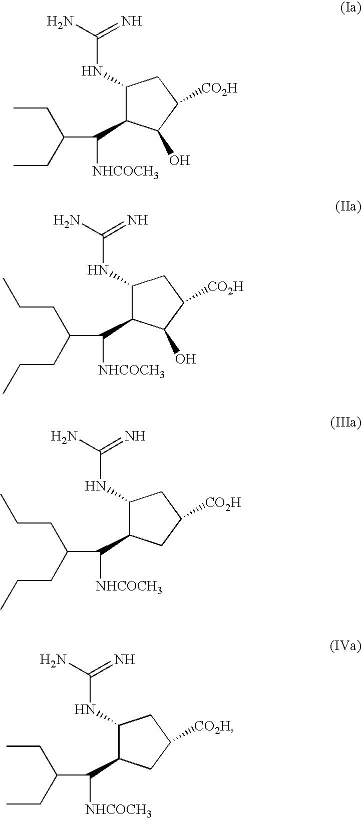

or a pharmaceutically acceptable salt thereof.

A specific compound of formula I, II, III, or IV is (1S,2S,3R,4R)-3-(1-Acetamido-2-ethylbutyl)-4-guanidino-2-hydroxycyclopentane-carboxylic acid; (1S,2S,3R,4R)-3-(1 -Acetamido-2-propylpentyl)-4-guanidino-2-hydroxycyclopentanecarboxylic acid; (1 R,3R,4R)-3 -(1 -Acetamido-2-propylpentyl)-4-guanidinocyclopentanecarboxylic acid; or (1R,3R,4R)-3-(1-Acetamido-2-ethylbutyl)-4-guanidinocyclopentanecarboxylic acid; or a pharmaceutically acceptable salt thereof.

A specific compound of formula I is a compound of formula Ia, or a pharmaceutically acceptable salt thereof.

It will be appreciated by those skilled in the art that compounds having one or more chiral centers may exist in and be isolated in optically active and racemic forms. Some compounds may exhibit polymorphism. It is to be understood that the present invention encompasses the use of any racemic, optically-active, polymorphic, or stereoisomeric form, or mixtures thereof, of a compound of formula I, II, III, and/or IV, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine anti-viral (e.g. anti-influenza) activity using the standard tests described herein, or using other similar tests which are well known in the art.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, phosphate, bicarbonate, and carbonate salts.

Pharmaceutically acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula I, II, III, and IV can be formulated as pharmaceutical compositions and administered to a mammalian host, such as a human patient, by intramuscular routes. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms. In some embodiments of the invention, the compounds of formula I, II, III, and/or IV are formulated with a buffer, e.g., a citrate, e.g., sodium citrate.

The pharmaceutical dosage forms suitable for injection or infusion can include sterile aqueous solutions or dispersions or sterile powders comprising the active ingredient(s) which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers (e.g., sodium citrate) or sodium chloride. Prolonged absorption of the injectable compositions can be brought about by the use in the compositions of agents delaying absorption, for example, aluminum monostearate and gelatin.

Sterile injectable solutions can be prepared by incorporating the active compound(s) into an appropriate solvent with the other optional ingredients, e.g., enumerated above, optionally followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

As used herein the terms “treat”, “treating” and “treatment” include administering a compound prior to the onset of clinical symptoms of a disease state/condition so as to prevent the development of any symptom, as well as administering a compound after the onset of one or more clinical symptoms of a disease state/condition so as to reduce or eliminate any such symptom, aspect or characteristic of the disease state/condition. Such treating need not be absolute to be useful. As illustrated hereinbelow, the active compounds can be administered prior to exposure to the virus. The agents can also be administered subsequent (e.g., within 1, 2, 3, 4, or 5 days) to exposure to the virus.

As used herein the term “unit dosage form” relates to an intramuscular formulation containing a specific amount of a drug (e.g., from about 10 mg to about 500 mg, e.g., about 150 mg), the whole of which is intended to be administered as a single dose. It is distinguished from a supply of an indefinite amount of a medicament, e.g., a bottle of medicine, from which a dose has to be measured out.

The invention will now be illustrated by the following non-limiting Example.

### EXAMPLE 1

### Intramuscular Treatment of Influenza with Peramivir

The efficacy of a single intramuscular injection of peramivir in the mouse influenza model was evaluated. To summarize, peramivir was found to be effective when administered intramuscularly in a mouse influenza virus infection model. Peramivir potently inhibited the neuraminidase enzyme N9 from H1N9 virus in vitro with a 50% inhibitory concentration (IC₅₀) of 1.3±0.4 nM. On-site dissociation studies indicated that peramivir remains tightly bound to N9 neuraminidase (t_{1/2}>24 h), whereas, zanamivir and oseltamivir carboxylate dissociate rapidly from the enzyme (t_{1/2}=1.25 h). A single intramuscular injection of peramivir (10 mg/kg) significantly reduced weight loss and mortality in mice infected with influenza A/H1N1, while oseltamivir demonstrated no efficacy by the same treatment regimen. Additional efficacy studies indicated that a single injection of peramivir (2-20 mg/kg) was comparable to an oral q.d.×5 day course of orally administered oseltamivir (2-20 mg/kg/day) in preventing lethality in H3N2 and H1N1 influenza models. Thus, a single intramuscular injection of peramivir can be used treat influenza infections and provides an alternate option to oseltamivir during an influenza outbreak.

Results

The ability of peramivir to inhibit the neuraminidase activity of N9 from the H1N9 virus was tested and compared to zanamivir and oseltamivir carboxylate. The IC₅₀ for peramivir (1.3±0.4 nM), oseltamivir carboxylate (2.1±0.4 nM), and zanamivir (1.6±0.3 nM) against N9 enzyme were not significantly different. The IC₉₀ values were: for peramivir 5.0±1.1 nM; for oseltamivir carboxylate 10.4±0.7 nM; and for zanamivir 10.0±1.2 nM.

In the mouse influenza model, viral infection leads to loss of body weight and high mortality, and this decrease in body weight correlates with pulmonary viral titer and pulmonary lesion score. Therefore, the efficacy of orally and intramuscular administered peramivir, oseltamivir and zanamivir were evaluated on the basis of the weight loss, mean days to death and survival rate, measured for 16 or 21 days post-infection for treated, infected animals relative to untreated, infected (control) animals.

In the prophylaxis model, a single intramuscular injection of peramivir, given 4 hours before viral challenge with H1N1 virus, was compared to an oral treatment of peramivir once daily for 5 days at doses of 1 and 10 mg/kg/day. Complete protection against lethality was observed in the mice treated at 10 mg/kg with both treatment regimens. However, at the 1 mg/kg dose, 60% of the mice survived in the oral treatment group versus 40% survival in the intramuscular treatment group. Mice treated with a single intramuscular injection of peramivir (10 mg/kg) demonstrated no weight loss by day 5, whereas mice given peramivir orally for 5 days at the same dose lost 0.22 g.

Peramivir was also administered at 2, 10, and 20 mg/kg as a single intramuscular injection 4 hours before viral infection with the H1N1 virus. Complete protection against lethality was observed at all doses. However, none of the five saline-treated control mice survived. By comparison, complete protection against lethality was also observed in the mice treated orally with oseltamivir at both 2 and 10 mg/kg/day (q.d.×5 days). No signs of drug-related toxicity were observed when peramivir was administered intramuscular at the highest dose (20 mg/kg).

Peramivir and oseltamivir showed a dose response relationship when the weight loss of infected mice over time was followed. At day 8, the maximum mean weight loss in the 2, 10, and 20 mg/kg peramivir-treated groups were 3.3, 0.98, and 0 g, respectively. Additionally, oseltamivir provided a similar effect with the greatest mean weight loss of 1.34 and 0 g occurring at day 8 for the 2 and 10 mg/kg groups, respectively. Day 5 weight loss shows a similar trend. In general, a lower dose resulted in greater weight loss when compared with a higher dose.

Single intramuscular injections of peramivir or oseltamivir at 10 mg/kg dose were evaluated when administered 4 hours prior to inoculation with H1N1 virus. Oseltamivir provided only 30% protection, which is not significantly different from the control group in which 90% of the mice died. In the peramivir-treated group, complete protection against lethality was observed. The peramivir group did not show any substantial weight loss (about 1.7% of initial weight). On the other hand, the oseltamivir group lost significant weight, about 4 g (25% of initial weight), and only 3 out of 10 mice survived. In the same model, a comparison of single oral treatments of a 10 mg/kg dose of either peramivir or oseltamivir was assessed. Peramivir provided better protection orally with a survival rate of 50%, whereas, only 10% of mice survived in the oseltamivir group.

To determine if similar protective effects are observed using different viruses, the efficacy of a single intramuscular injection of peramivir was compared to the oral treatment of oseltamivir (q.d.×5 days) in mice infected with the H3N2 virus. In this study the drug was administered 1 hour prior to viral inoculation. The single intramuscular treatment of peramivir at a 20 mg/kg dose provided almost complete protection against lethality (9/10 survived). Oseltamivir also demonstrated similar protective effects (9/10 survived). The mean weight loss was almost identical in both treatment groups in that the oseltamivir treated mice lost 28% of their weight (about 5.1 g) compared to 25% (4.5 g) weight loss in the peramivir group by day 8. In the delayed treatment model, intramuscular administration of a 10 mg/kg single dose of peramivir 24 hours or 48 hours post-infection gave complete protection against lethality, whereas, in the saline group, 70% lethality was observed. There was no significant weight loss by day 5 in both 24 and 48 hours peramivir-treated groups given both orally (q.d.×5 days) and by single intramuscular injection, whereas, the saline-treated group lost 2.1 g.

Materials and Methods

The influenza A viruses used in this study were obtained from American Type Culture Collection, Manassas, Va., USA (A/NWS/33;H1N1) and Dr. Robert Sidwell, Utah State University, Logan, Utah, USA (A/Victoria/3/75;H3N2) and were mouse adapted. Purified N9 crystals from A/H1N9 (NWS/G70) avian virus were obtained from Dr. Graeme Laver, Australian National University, Canberra, Australia.

Specific pathogen-free female BALB/c mice (10-19 g) were obtained from Charles Rivers Laboratories (Raleigh, N.C., USA). They were quarantined for 24 hours prior to infection and maintained on rodent diet from Harlan Teklad and tap water.

Peramivir, oseltamivir, oseltamivir carboxylate and zanamivir were synthesized by BioCryst Pharmaceuticals, Inc. (Birmingham, Ala., USA). Each compound was prepared in sterile 0.9% sodium chloride for in vivo experiments. A mixture of 5% isoflurane/95% oxygen was administered as anesthesia.

A standard fluorimetric assay was used to measure influenza virus neuraminidase activity (Potier et al., Anal. Biochem., 94, 287-296 (1979)). The substrate (2′-(4-methylumbelliferyl)-α-D-acetylneuraminic acid, MuNANA) is cleaved by neuraminidase to yield a fluorescent product that can be quantified. The assay mixture contained inhibitor at various concentrations and neuraminidase enzyme in 32.5 mM MES (2-(N-morpholino)- ethanesulfonic acid) buffer, 4 mM calcium chloride at pH6.5 and incubated for 10-30 min. The reaction was started by the addition of the substrate. After incubation for 30-120 min fluorescence was recorded (excitation: 360 nm and emission: 450 nm) and substrate blanks were subtracted from the sample readings. The IC₅₀ was calculated by plotting percent inhibition of neuraminidase activity versus the inhibitor concentration. The results are reported as the average of three experiments.

Mice were anesthetized with isoflurane and exposed to 100 μL of virus by intranasal instillation. In the prophylaxis model, drug was administered 1 or 4 hours before viral infection; in the treatment model, drug was given at times indicated after the viral infection. Each infected, drug and saline-treated group contained 5-10 mice. All mice were observed daily for changes in weight and for any deaths. Parameters for evaluation of antiviral activity included weight loss, reduction in mortality and/or increase in mean days to death determined through 16or21 days.

Mice were infected intranasally with an approximately 70-90% lethal dose of the A/NWS/33(H1N1) or A/Victoria/3/75 (H3N2) influenza virus. Oral treatment with peramivir or oseltamivir (prepared in injection-grade saline) began 1 or 4 hours before virus exposure (prophylaxis model) and continued once daily for 5 days unless indicated. A single intramuscular treatment was administered 1 or 4 hours before virus exposure or at times indicated (treatment model). Normal and saline-treated control mice were included in the same treatment schedule. Parameters studied were reduction in mortality and/or increase in mean days to death.

The data was analyzed by Sigma Plot (Windows Version 4.01, SPSS, Chicago, Ill., USA) and Sigma Stat (Windows Version 2.0, Jandel Corporation, San Rafael, Calif., USA). The t-test was used to evaluate differences in mean days to death. One-way analysis of variance (ANOVA) was performed using the Holm-Sidak test for pairwise multiple comparisons to evaluate differences in weight loss. Kaplan-Meier survival analysis (log rank or Gehan-Breslow tests) were applied to survival number differences.

These findings, including methods, results, and discussion, are provided in Bantia et al., Antiviral Research, 69, 39-45 (2006).

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed.

What is claimed is:

1. A method for treating a viral infection in a human comprising administering an effective anti-viral amount of a compound of formula I, II, III, or IV: 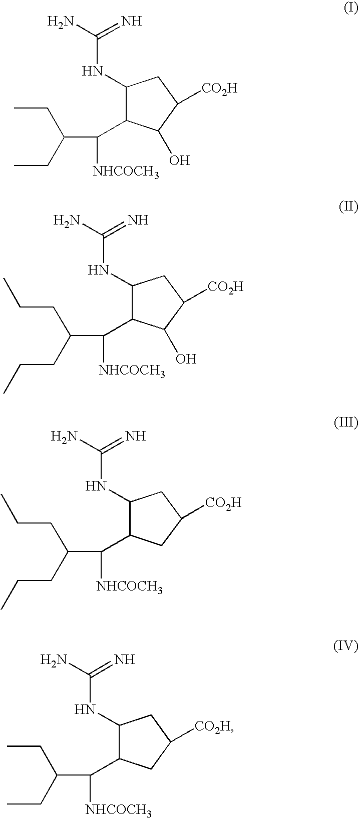
or a pharmaceutically acceptable salt thereof, to the human by an intramuscular route.

2. The method of claim 1, wherein the compound of formula I, II, III, or IV is a compound of formula Ia, IIa, IIIa, or IVa: 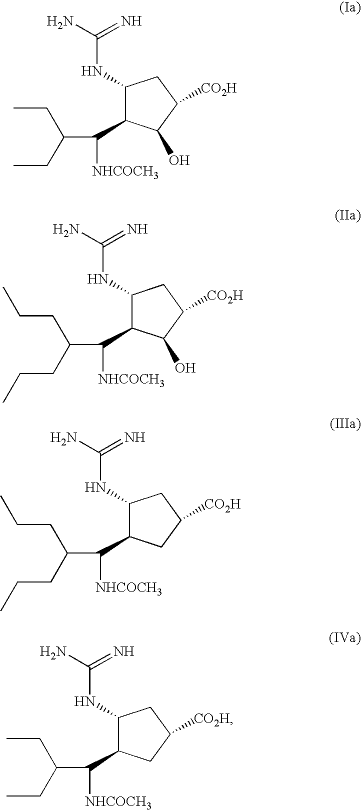
or a pharmaceutically acceptable salt thereof.

3. The method of claim 1, wherein the viral infection is an influenza infection.

4. The method of claim 3, wherein the viral infection is an influenza type A or type B infection.

5. The method of claim 3, wherein the influenza is an H3N2, H1N1, H5N1, avian, or seasonal influenza.

6. The method of claim 1, wherein the effective anti-viral amount is up to about 500 mg.

7. The method of claim 1, wherein the effective anti-viral amount is up to about 150 mg.

8. The method of claim 1, wherein the effective anti-viral amount is about 150 mg.

9. The method of claim 1, wherein the entire effective dose is administered in one intramuscular administration.

10. The method of claim 2, wherein a compound of formula Ia, or a pharmaceutically acceptable salt thereof, is administered.

11. The method of claim 1, further comprising orally administering a neuraminidase inhibitor to the human.

12. The method of claim 11, wherein the neuraminidase inhibitor that is administered orally is oseltamivir carboxylate.

13. The method of claim 11, wherein the neuraminidase inhibitor that is administered orally is a compound of formula I, II, III, or IV: 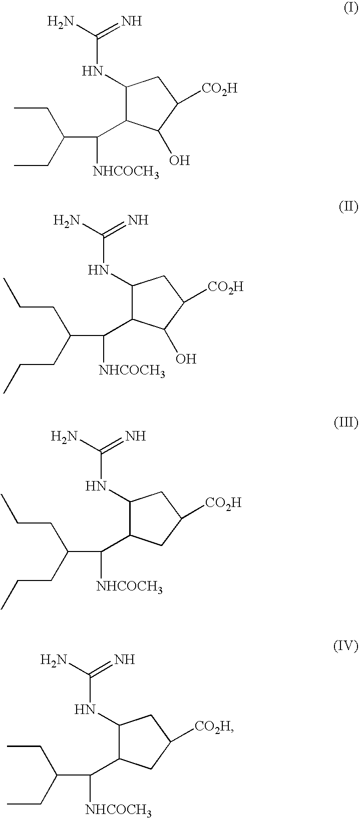
or a pharmaceutically acceptable salt thereof.

14. The method of claim 11, wherein the neuraminidase inhibitor that is administered orally is a compound of formula Ia, Ia, IIIa, or IVa: 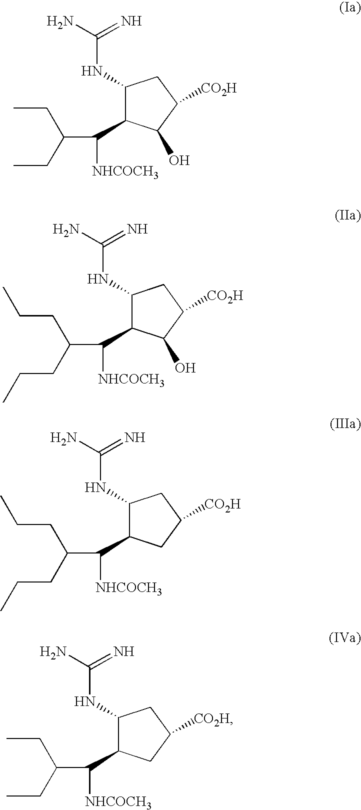
or a pharmaceutically acceptable salt thereof.

15. The method of claim 14, wherein the neuraminidase inhibitor that is administered orally is a compound of formula Ia, or a pharmaceutically acceptable salt thereof.

16. The method of claim 11, wherein the neuraminidase inhibitor that is administered orally is administered for up to 20 days.

17. The method of claim 16, wherein the neuraminidase inhibitor that is administered orally is administered for up to 10 days.

18. The method of claim 17, wherein the neuraminidase inhibitor that is administered orally is administered for up to 5 days.

19. The method of claim 1, wherein the effective anti-viral amount is an amount effective to increase the life expectancy of the human.

20. A method for inhibiting a neuraminidase in a human comprising administering an effective inhibitory amount of a compound of formula I, II, III, or IV: 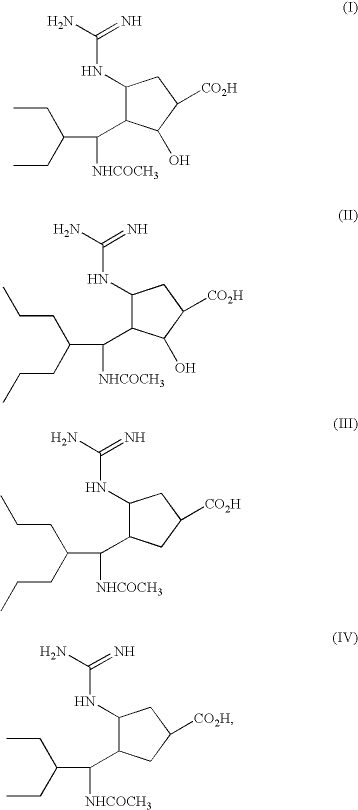
or a pharmaceutically acceptable salt thereof, to the human by an intramuscular route.

21. The method of claim 20, wherein the compound of formula I, II, III, or IV is a compound of formula Ia, IIa, IIIa, or IVa: 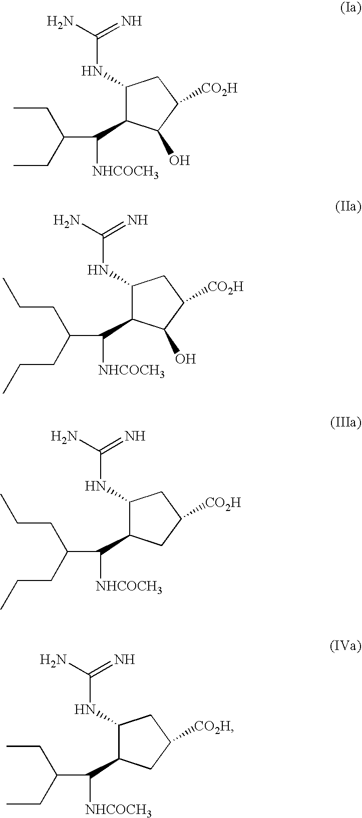
or a pharmaceutically acceptable salt thereof.

22. The method of claim 20, wherein the effective inhibitory amount is up to about 500 mg.

23. The method of claim 20, wherein the effective inhibitory amount is up to about 150 mg.

24. The method of claim 20, wherein the effective inhibitory amount is about 150 mg.

25. The method of claim 20, wherein the entire effective inhibitory dose is administered in one intramuscular administration.

26. The method of claim 20, wherein a compound of formula Ia, or a pharmaceutically acceptable salt thereof, is administered.

27. The method of claim 20, further comprising orally administering a neuraminidase inhibitor to the human.

28. The method of claim 27, wherein the neuraminidase inhibitor that is administered orally is oseltamivir carboxylate.

29. The method of claim 27, wherein the neuraminidase inhibitor that is administered orally is a compound of formula I, II, III, or IV: 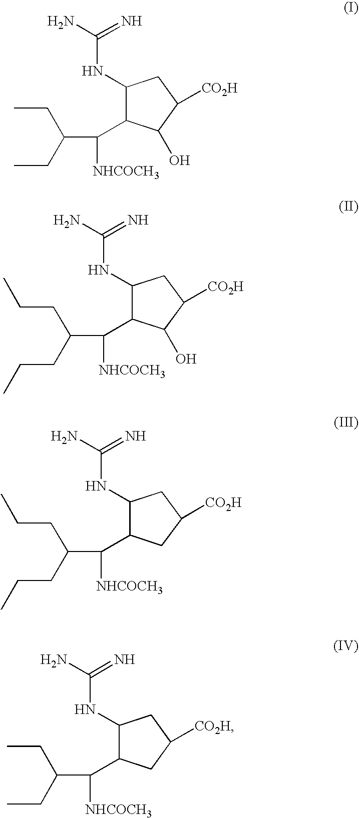
or a pharmaceutically acceptable salt thereof.

30. The method of claim 27, wherein the neuraminidase inhibitor that is administered orally is a compound of formula Ia, IIa, IIIa, or IVa: 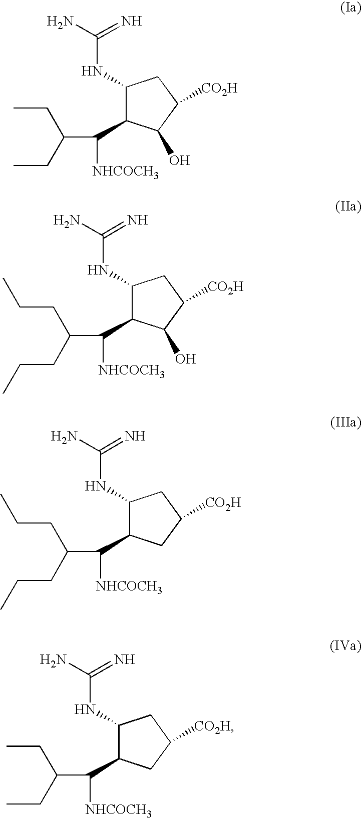
or a pharmaceutically acceptable salt thereof.

31. The method of claim 30, wherein the neuraminidase inhibitor that is administered orally is a compound of formula Ia, or a pharmaceutically acceptable salt thereof.

32. The method of claim 27, wherein the neuraminidase inhibitor that is administered orally is administered for up to 20 days.

33. The method of claim 32, wherein the neuraminidase inhibitor that is administered orally is administered for up to 10 days.

34. The method of claim 33, wherein the neuraminidase inhibitor that is administered orally is administered for up to 5 days.

35. The method of claim 20, wherein the effective inhibitory amount is an amount effective to increase the life expectancy of the human.

36. A unit dosage form that is suitable for intramuscular administration to a human, comprising up to about 500 mg of a compound of formula I, II, III, or IV: 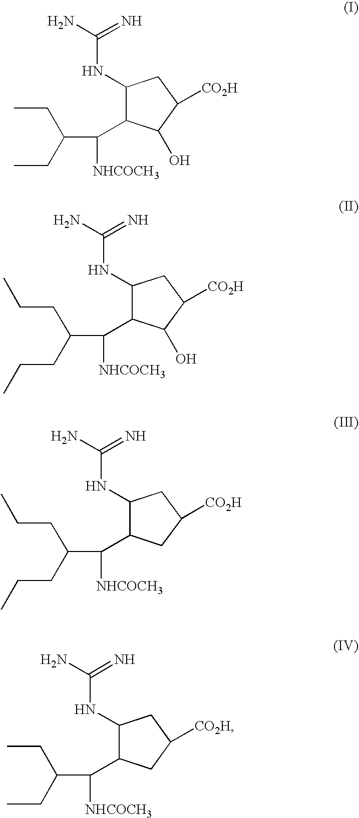
or a pharmaceutically acceptable salt thereof.

37. The unit dosage form of claim 36, wherein the compound of formula I, II, III, or IV is a compound of formula Ia, IIa, IIIa, or IVa: 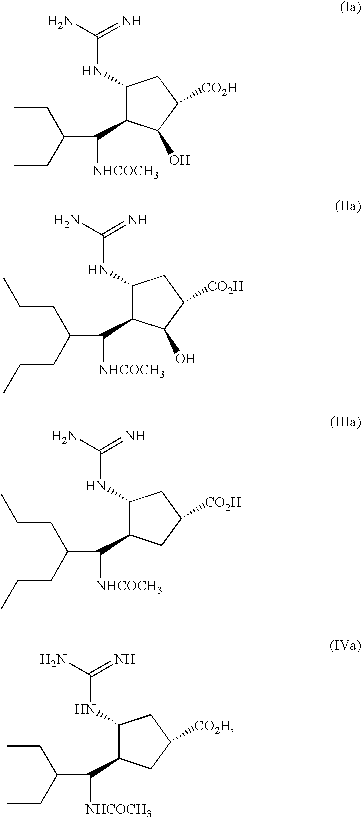
or a pharmaceutically acceptable salt thereof.

38. The unit dosage form of claim 36 that comprises about 150 mg of the compound or salt.

39. A kit, comprising packaging materials, a unit dosage form as described in claim 36, and instructions for administering the unit dosage form to a human by an intramuscular route.
